(19) United States
(12) Patent Application Publication
(10) Pub. No.: US 2010/0298629 A1
(43) Pub. Date: Nov. 25, 2010

(54) DEVICE AND METHOD FOR SUSPENDING AN ORGAN DURING SURGERY

(76) Inventor: Chih-Kun HUANG, Kaohsiung County (TW)

Correspondence Address: LADAS & PARRY LLP, 224 SOUTH MICHIGAN AVENUE, SUITE 1600, CHICAGO, IL, 60604, US

(21) Appl. No.: 12/469,004

(22) Filed: May 20, 2009

(51) Int. Cl. A61B 17/00

(52) U.S. Cl. 600/37

(57) ABSTRACT

An organ suspending device is adapted for suspending an organ in a patient's body cavity during surgery, and includes an organ holding member and a suspending member. The organ holding member is adapted to be disposed in the patient's body cavity so as to hold the organ. The suspending member is adapted to be disposed in the patient's body cavity, and has two hanging elements and two piercing elements. The organ holding member together with the organ can be pulled in a direction toward a cavity wall of the patient's body cavity and away from its original position when the piercing elements are pulled outwardly of the cavity wall.

A method for traction of an organ during surgery employs the organ suspending device and is also disclosed.

### BACKGROUND OF THE INVENTION

1. Field of the Invention

This invention relates to a suspending device and a suspending method, more particularly to a device and a method for suspending an organ during a surgical operation.

2. Description of the Related Art

In Asia, Roux-en-Y gastric bypass is a rising surgical operation applied to reduce weight. Most studies have shown that about 60-70% of excess body weight can be reduced after the surgical operation. Furthermore, the effect of the surgical operation can be maintained for over 10 years. A laparotomy is conducted to gain access into a patient's abdominal cavity for performing the Roux-en-Y gastric bypass. Therefore, the surgical operation gives rise to a long abdominal wound. The patient may feel more pain due to the wound and may need prolonged time for a full recovery from the surgical operation. After the wound heals, a scar forms and hence affects the physical appearance of the patient.

In view of the aforementioned drawback of the Roux-en-Y gastric bypass, Wittgrove et. al. proposed the use of a laparoscope to perform laparoscopic Roux-en-Y gastric bypass (LRYGB) in 1993. Compared to the prior Roux-en-Y gastric bypass that includes the laparotomy, when LRYGB is conducted, only about 5 to 7 incisions of nearly 1 cm are necessary to be made in an abdominal wall of a patient. LRYGB can be accomplished using laparoscopic surgical instruments. Consequently, long abdominal wounds and complications such as wound dehiscence, infection, and pain are prevented. However, the patient demanding LRYGB to reduce weight is generally obese and therefore has a hypertrophic liver that covers a stomach thereof. In this case, LRYGB is made more difficult to perform.

Referring to FIG. 1, besides disposing the laparoscopic surgical instruments in the patient's abdominal cavity through the 5-7 incisions in the patient's abdominal wall, a conventional liver retractor 11 is placed in the patient's abdominal cavity via one of the incisions so as to lift up the liver and obtain a clear view of the stomach, thereby facilitating LRYGB.

Nevertheless, the liver retractor 11 is normally attached to a retract-robot 12 (produced by Arrow Medical Supply Inc.) that is disposed on an operation table and that takes up room due to a large size thereof. As a whole, the patient has 5-7 abdominal wounds after LRYGB that utilizes the liver retractor 11 is conducted. Nowadays, minimally invasive surgery is a trend. Minimizing possible wounds of a patient during a surgical operation is a goal in the medical and related fields. Thus, a device useful to achieve the goal is needed.

### SUMMARY OF THE INVENTION

Therefore, the object of the present invention is to provide a device and a method for suspending an organ in order to minimize possible wounds of a patient.

According to one aspect of this invention, there is provided an organ suspending device. The device is adapted for suspending an organ in a patient's body cavity having a cavity wall during surgery, and includes an organ holding member and a suspending member. The organ holding member is adapted to be disposed in the patient's body cavity so as to hold the organ. The suspending member is adapted to be disposed in the patient's body cavity, and has two hanging elements that respectively hang two opposite ends of the organ holding member, and two piercing elements that can pierce the cavity wall and that are connected respectively to the hanging elements.

The piercing elements can pierce and extend out of the cavity wall after the organ holding member and the suspending member are placed within the patient's body cavity. The organ holding member together with the organ can be pulled in a direction toward the cavity wall and away from its original position when the piercing elements are pulled outwardly of the cavity wall.

According to another aspect of this invention, there is provided an organ suspending device. The device is adapted for suspending an organ in a patient's body cavity having a cavity wall during surgery, and includes an organ holding member and a suspending member. The organ holding member is adapted to be disposed in the patient's body cavity so as to hold the organ. The suspending member is adapted to be disposed in the patient's body cavity, hangs the organ holding member, and has at least one piercing element that can pierce the cavity wall.

The piercing element can pierce and extend out of the cavity wall after the organ holding member and the suspending member are placed within the patient's body cavity. The organ holding member together with the organ can be pulled in a direction toward the cavity wall and away from its original position when the piercing element is pulled outwardly of the cavity wall.

According to yet another aspect of this invention, there is provided a method for traction of an organ during surgery. The method comprises: placing an organ suspending device into a patient's body cavity that has a cavity wall; attaching the organ suspending device to an organ; manipulating the organ suspending device to pierce the cavity wall, and pulling a portion of the organ suspending device outwardly of the cavity wall so that the organ is pulled in a direction toward the cavity wall and away from its original position; and immobilizing the portion of the organ suspending device on the cavity wall at an outside of the cavity wall, thereby suspending the organ within the patient's body cavity.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a perspective view of a conventional liver retractor attached to a retract-robot;

FIG. 2 is a schematic diagram of the first preferred embodiment of an organ suspending device according to this invention;

FIG. 3 is a schematic diagram illustrating how the organ suspending device shown in FIG. 2 is used for hanging a liver; and

FIG. 4 is a schematic diagram of the second preferred embodiment of the organ suspending device according to this invention.

### DETAILED DESCRIPTION OF THE PREFERRED EMBODIMENTS

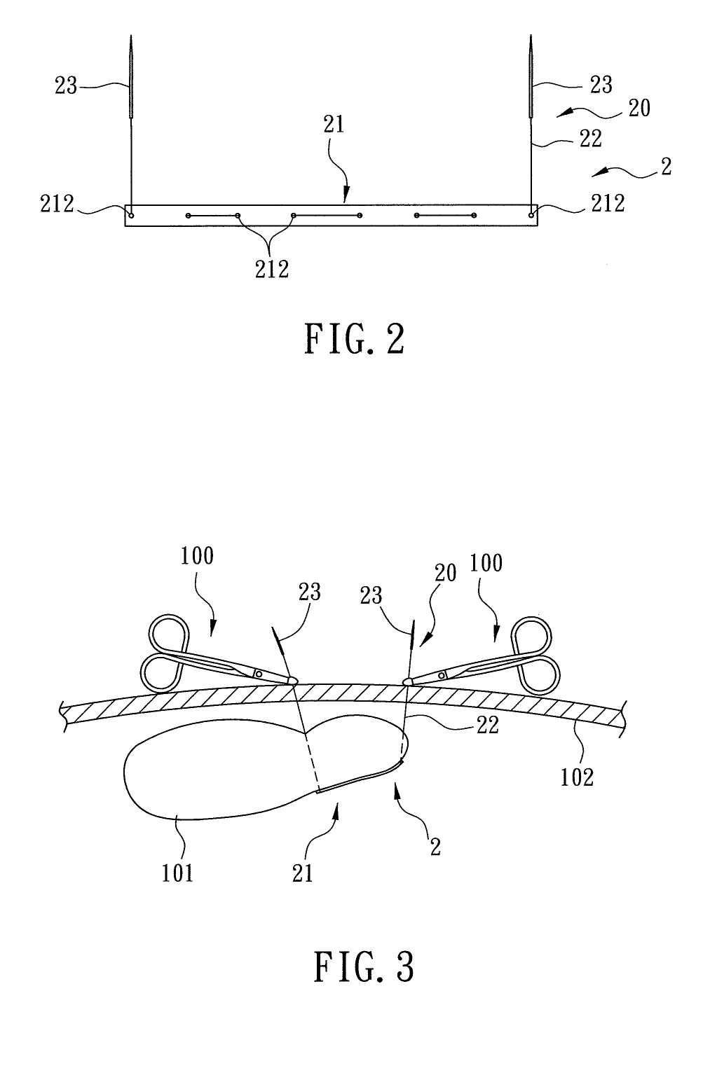

Referring to FIGS. 2 and 3, the first preferred embodiment of an organ suspending device 2 according to the present invention is adapted for suspending an organ 101 in a patient's body cavity during surgery, and includes an organ holding member 21 and a suspending member 20. The organ holding member 21 is adapted to be disposed in the patient's body cavity so as to hold the organ 101.

The suspending member 20 is connected to the organ holding member 21 and has two piercing elements 23 that are capable of piercing a cavity wall 102 of the patient's body cavity. Preferably, the suspending member 20 further has two hanging elements 22 that respectively hang two opposite ends of the organ holding member 21 and that are respectively connected to the piercing elements 23. The piercing elements 23 can pierce and extend out of the cavity wall 102 after the organ holding member 21 and the suspending member 20 are placed within the patient's body cavity. The organ holding member 21 together with the organ 101 can be pulled in a direction toward the cavity wall 102 and away from an original position of the organ 101 when the piercing elements 23 are pulled outwardly of the cavity wall 102.

In this embodiment, the organ suspending device 2 is used for suspending a patient's liver 101 in an abdominal cavity during laparoscopic Roux-en-Y gastric bypass (LRYGB), thereby lifting the liver 101 away from the original position thereof, and resulting in a clear view of the patient's stomach (not shown) to facilitate LRYGB. The abdominal cavity has an abdominal wall 102. An immobilizing unit that includes two hemostatic clamps 100, such as surgical Kelly clamps, is disposed outside the abdominal cavity to clamp the hanging elements 22 of the suspending member 20 on the abdominal wall 102.

Preferably, the organ holding member 21 is an elongate flat flexible member that has the two opposite ends thereof connected respectively to the hanging elements 22 of the suspending member 20, and that has a plurality of through-holes 212. Two of the through-holes 212 are disposed at the two opposite ends of the organ holding member 21, and the remaining through-holes 212 are between the two opposite ends. A length of the organ holding member 21 can be varied according to different demands. A lay-flat Jackson-Pratt drain tube generally used in surgery can be used as the organ holding member 21 because the drain tube has proper strength and length sufficient to hold an organ, and is originally provided with drain holes that can be used as the through-holes 212. When a portion of the Jackson-Pratt drain tube is cut off for use as the organ holding member 21, the cut portion will have two through-holes on two opposite ends thereof and other through-holes between the two opposite ends.

In this embodiment, the hanging elements 22 are made of a single thread that is extended through the through-holes 212 between the two ends of the organ holding member 21 using one of the piercing elements 23, and that has two end sections extending respectively and outwardly from the two ends of the organ holding member 21 to be used as the hanging elements 22. The single thread may be obtained from a surgical suture. The piercing elements 23 of the suspending member 20 may be surgical needles. Generally, a surgical needle is already connected integrally to a surgical suture when purchased. In this embodiment, a surgical suture having two ends connected to two surgical needles (a product of Ethicon Inc., 2-O Prolene suture, Model No. Monofilament Polypropylene Suture W8400) is used as the suspending member 20.

A method for traction of an organ during surgery according to a preferred embodiment of the present invention employs the organ suspending device 2. The method includes the following steps. Initially, the organ suspending device 2 is first placed into a patient's body cavity, which has a cavity wall 102. Then, the organ suspending device 2 is attached to an organ 101. Thereafter, the organ suspending device 2 is manipulated to pierce the cavity wall 102, and a portion of the organ suspending device 2 is pulled outwardly of the cavity wall 102 so that the organ 101 is pulled in a direction toward the cavity wall 102 and away from its original position. When the portion of the organ suspending device 2 is immobilized on the cavity wall 102 at an outside of the cavity wall 102, the organ 101 is suspended within the patient's body cavity.

Preferably, the portion of the organ suspending device 2 is immobilized by using two hemostatic clamps on the cavity wall 102. Furthermore, the organ suspending device 2 is attached to the organ 101 by causing the organ suspending device 2 to penetrate through the organ 101 and by placing another portion of the organ suspending device 2 around and in abutment with a part of the organ 101. In addition, the organ suspending device 2 is placed in the patient's body cavity by passing the same through a trocar.

While two piercing elements 23 are used in the preferred embodiment of the present invention, the number of the piercing elements 23 should not be limited to two. The number of the piercing elements 23 may be one as long as the organ holding member 21 with the organ 101 can be hung by the hanging elements 22 within the patient's body cavity when the suspending member 20 is immobilized on the cavity wall 102. For example, one piercing element 23 may be connected to one of the hanging elements 22 which in turn is connected to the other hanging element 22 so that, when the piercing element 23 is pulled outwardly of the cavity wall 102, the hanging elements 22 can hang the organ holding member 21 and the organ 101.

### EXAMPLE

An example of the method for traction of an organ is described as follows. The example is directed to a method for traction of a liver within the patient's abdominal cavity via the organ suspending device during LRYGB. A single incision was made around the patient's umbilicus so as to insufflate the abdominal cavity with carbon dioxide (i.e., pneumoperitoneum). A first trocar (15 mm VersaStep™ Bladeless Trocar produced by Covidien) was placed at the incision. A rigid video laparoscope that had a diameter of 5 mm, that had a fixed 30° view angle, and that can transmit images was disposed in the abdominal cavity through the first trocar.

Normally, the patient requiring LRYGB is obese and consequently has the hypertrophic liver with a left lobe covering the stomach. As a result, traction of the left lobe of the liver is required during LRYGB so as to result in a clear view of the stomach for performing LRYGB. In the example, a length of the left lobe of the liver was measured by means of the image transmitted from the laparoscope, and a section of the Jackson-Pratt drain tube near a drainage hole site was cut with a length fitting the length of the left lobe.

Afterwards, 2-0 Prolene suture having two needles was used to penetrate through the drainage holes of the cut section of the drain tube. Thus, the organ suspending device 2 according to the present invention was assembled.

Two second trocars (VERSAPORT™ V2 5 mm Trocar with Fixation Cannula produced by Covidien) were respectively placed at two ends of the incision. The organ suspending device 2 was placed in the abdominal cavity via one of the second trocars.

By means of the image transmitted from the laparoscope, one of the needles was manipulated using a needle holder to pierce a portion of the left lobe of the liver, which was adjacent to a falciform ligament of the liver, and subsequently pierce the midline of the abdominal wall. The other needle was manipulated by dint of the needle holder to pierce a lateral edge of the left lobe of the liver, and then to pierce a left upper quadrant of the abdominal wall. Thereafter, the two needles were manipulated to pierce and extend out of the abdominal wall. When the needles were pulled outwardly of the abdominal wall, the liver was pulled in a direction toward the abdominal wall and away from its original position. When two end sections of the suture connected to the needles were clamped by two Kelly clamps, the liver was suspended within the abdominal cavity. The video image transmitted from the laparoscope was helpful to suspend the liver at an appropriate position. Afterward, LRYGB can be proceeded. Since the feature of this invention does not reside in the installation and operative methods with respect to the first and second trocars, and the laparoscope, and procedures of LRYGB, further details of the same are omitted herein for the sake of brevity.

Some of the advantages of the organ suspending device 2 are as follows:

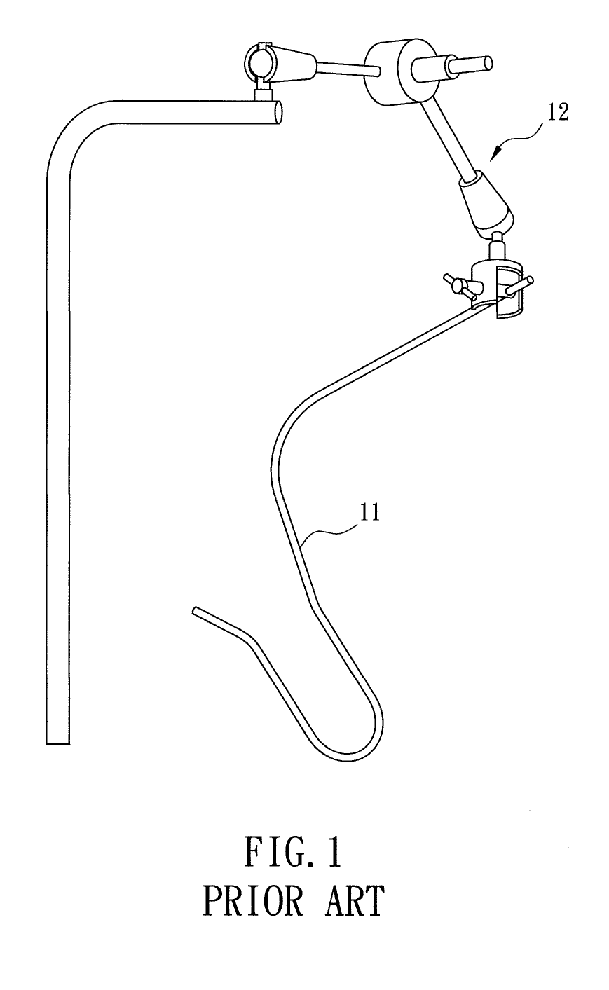

1. The organ suspending device 2 has a simple structure that is capable of suspending an organ 101. Since the organ holding member 21 is flat, the organ 101 is not easily removed from the organ holding member 21 when the organ holding member 21 supports the organ 101. During surgery, the conventional liver retractor 11 and the retract-robot 12 (see FIG. 1) are not required to lift the organ 101.

2. Compared to the conventional liver retractor 11, the organ suspending device 2 has a much smaller size such that the organ suspending device 2 can be disposed in the abdominal cavity through the single incision that is also used for disposing the first and second trocars, the laparoscope, and other laparoscopic surgical instruments. A separate and exclusive incision of about 1 cm is necessary for the conventional liver retractor 11. No such incision is required for the organ suspending device 2. The piercing elements 23 only result in two small wounds that are like wounds arising from syringe needles, thereby efficiently reducing the patient's pain after the surgery, shortening the time for full recovery from the surgery, and strongly lowering the possibility of inducing abdominal scars.

3. Since the organ suspending device 2 is simply made from widely used surgical needles and suture, the production cost of the organ suspending device 2 is lower than that of the combination of the conventional liver retractor 11 and the retract-robot 12. Moreover, the organ suspending device 2 only takes up very small space.

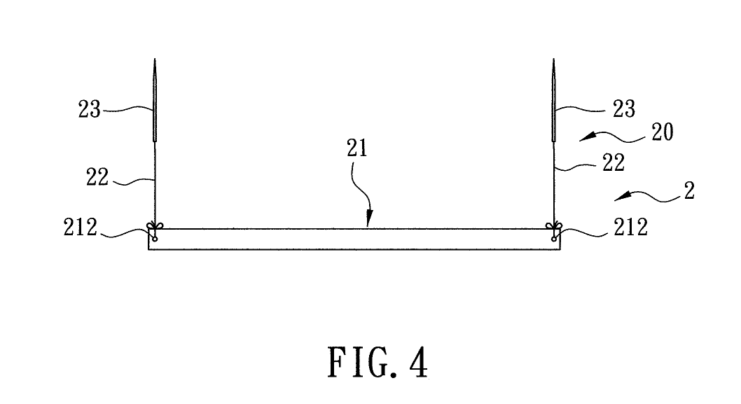

Referring to FIG. 4, the second preferred embodiment of the organ suspending device 2 according to the present invention is similar to the first preferred embodiment except that the hanging elements 22 are made from two threads which are respectively attached to the two ends of the organ holding member 21, and that the organ holding member 21 only has the two through-holes 212 on the two opposite ends thereof. More specifically, each of the threads is passed through a respective one of the two through-holes 212 using a respective one of the piercing elements 23, and is tied so as to be fixedly connected to the organ holding member 21.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. An organ suspending device adapted for suspending an organ in a patient's body cavity during surgery, the patient's body cavity having a cavity wall, said organ suspending device comprising:
an organ holding member adapted to be disposed in the patient's body cavity so as to hold the organ; and
a suspending member adapted to be disposed in the patient's body cavity, and having two hanging elements that respectively hang two opposite ends of said organ holding member, and two piercing elements that can pierce the cavity wall and that are connected respectively to said hanging elements;
wherein said piercing elements can pierce and extend out of the cavity wall after said organ holding member and said suspending member are placed within the patient's body cavity, and wherein said organ holding member together with the organ can be pulled in a direction toward the cavity wall and away from its original position when said piercing elements are pulled outwardly of the cavity wall.

2. The organ suspending device as claimed in claim 1, wherein said organ holding member has a plurality of through-holes, said hanging elements being made of a single thread that extends through said through-holes and that has two end sections extending respectively and outwardly from said two ends of said organ holding member to be used as said hanging elements.

3. The organ suspending device as claimed in claim 1, wherein said hanging elements are respectively made from two threads which are attached to said two ends of said organ holding member, respectively.

4. The organ suspending device as claimed in claim 1, wherein said piercing elements are needles.

5. The organ suspending device as claimed in claim 1, further comprising an immobilizing unit to immobilize said hanging elements on the cavity wall.

6. The organ suspending device as claimed in claim 5, wherein said immobilizing unit includes two hemostatic clamps to clamp said hanging elements, respectively.

7. The organ suspending device as claimed in claim 1, wherein said organ holding member is flat, elongated, and flexible.

8. An organ suspending device adapted for suspending an organ in a patient's body cavity during surgery, the patient's body cavity having a cavity wall, said organ suspending device comprising:
an organ holding member adapted to be disposed in the patient's body cavity so as to hold the organ; and
a suspending member adapted to be disposed in the patient's body cavity, said suspending member hanging said organ holding member and having at least one piercing element that can pierce the cavity wall;
wherein said piercing element can pierce and extend out of the cavity wall after said organ holding member and said suspending member are placed within the patient's body cavity, and wherein said organ holding member together with the organ can be pulled in a direction toward the cavity wall and away from its original position when said piercing element is pulled outwardly of the cavity wall.

9. The organ suspending device as claimed in claim 8, wherein said organ holding member is flexible and flat, said piercing element is a needle, and said suspending member further has a thread that is connected to said needle and that hangs said organ holding member.

10. A method for traction of an organ during surgery, comprising:
placing an organ suspending device into a patient's body cavity that has a cavity wall;
attaching the organ suspending device to an organ;
manipulating the organ suspending device to pierce the cavity wall, and pulling a portion of the organ suspending device outwardly of the cavity wall so that the organ is pulled in a direction toward the cavity wall and away from its original position; and
immobilizing the portion of the organ suspending device on the cavity wall at an outside of the cavity wall, thereby suspending the organ within the patient's body cavity.

11. The method of claim 10, wherein the portion of the organ suspending device is immobilized by using a hemostatic clamp to clamp the portion of the organ suspending device on the cavity wall.

12. The method of claim 10, wherein the organ suspending device is attached to the organ by causing the organ suspending device to penetrate through the organ, and by placing another portion of the organ suspending device around and in abutment with a part of the organ.

13. The method of claim 10, wherein the organ suspending device is placed in the patient's body cavity by passing the same through a trocar.
